(19) United States
(12) Patent Application Publication
(10) Pub. No.: US 2009/0281518 A1
(43) Pub. Date: Nov. 12, 2009

(54) DEVICE FOR AUTOMATIC ADJUSTMENT OF THE DOSE OF MELATONIN AND/OR DELIVERY OF MELATONIN

(75) Inventors: Hendrika C. Krijnsen, Boxtel (NL); Ventzeslav P. Iordanov, Valkenswaard (NL); Geert Langereis, Eindhoven (NL); Michel P.B. Van Bruggen, Helmond (NL); Paul A.J. Ackermans, Neunen (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/440,590

(22) PCT Filed: Sep. 4, 2007
(86) PCT No.: PCT/IB07/53553

§ 371 Date: Mar. 10, 2009

(30) Foreign Application Priority Data
Sep. 13, 2006 (EP) 06120569.6

(51) Int. Cl. A61M 37/00

(52) U.S. Cl. 604/503; 604/66

(57) ABSTRACT

The invention relates to a device for melatonin treatment involving an adapting means which adapts the daily dose upon measurement of a body parameter of the patient or the user of the device.

This invention is in the field of devices for automatic adjustment of the dose of melatonin and/or delivery of melatonin

Melatonin (N-acetyl-5-methoxytryptamine) is a hormone synthesized and secreted by the pineal gland. The exact function of the hormone in adult human beings has not been determined. In healthy young adults, melatonin is secreted as a broad pulse during nighttime sleep in the total amount of approximately 25-30 μg per night, producing peak plasma concentrations of approximately 70 pg/ml, occurring at approximately 02:00 am. Melatonin is secreted into the blood stream and possibly also into cerebrospinal fluid (CSF) simultaneously. The terminal plasma elimination half-life is approximately 45 minutes; volume of distribution is approximately 40 liters; and the metabolic clearance of melatonin is approximately 1 liter per minute

The relationship of the melatonin cycle to the activity/rest (sleep) cycle is not clear, but it is believed that melatonin organizes at least partially the normal sleep pattern.

Melatonin has been given to human beings to treat the phenomenon of “jet lag” following airplane trips associated with a change in time zones (Arendt et al. (1987) Ergonomics 30:1379-1393); U.S. Pat. Nos. 4,600,723 and 4,665,086). It has been given to patients with Parkinson disease (Anton-Tay et al. (1971) Life Sciences 10:841-850), epilepsy (Anton-Tay et al., ibid.), or seasonal affective disorders (Wirz-Justice et al. (1990) J. Psychiat. Res. 24(2) :129-137). It has been tried as a sleep-wake organizer in desynchronized blind persons (Arendt et al. (1988) Lancet pp 772-773; Folkard et al. (1990) Neuroscience Lett. 113:193-198; Sack et al., (1987) in, “Temporal Disorder in Human Oscillatory Systems”, Eds. L. Rensing et al., Springer-Verlag, Heidelberg, pp 219-224; Sack and Lewy (1988) Am. Psychiatric Assoc. 141 Ann. Meeting, Montreal, Quebec). Improved timing of sleep cycles resulted. Oral melatonin has been given to insomniacs (Waldhouser et al. (1990) Psychopharmacology 100:222-226; Arendt et al. (1991) Lancet 337:1121-1124). Melatonin reduced the time awake before sleep onset and diminished sleep latency and number of awakenings. Overall sleep efficiency was increased, and mood, drive, alertness, and reaction time were improved the next day. Most of the published studies are consistent with the assumption that melatonin has mild sedative and hypnotic properties and may be a natural, sleep-inducing and sleep-organizing signal in humans. In addition, melatonin has been reported to lower intraocular pressure in glaucoma, to inhibit breast cancer (U.S. Pat. No. 4,855,305; Barch et al. (1991) Cancer 67:1681-1684), to be useful in the treatment of premenstrual depression (U.S. Pat. No. 4,945,103; Parry et al. (1990) Arch. Gen. Psychiatry 47:1139-1146; Yen et al. (1990) Arch. Gen. Psych. 47:1139-1146), for affecting contraception in humans (PCT Appln. WO 90/14084), and to prevent sudden infant death syndrome (Wurtman et al. (1990) Forensic Science Interntl. 45:171-180).

However, especially since melatonin is also an endogenous compound, it has been found that the optimal dosage is often difficult and the effect of under and/or overdosage often occurs. This is at least in part due to the circadian rhythms that trigger the anabolism and catabolism of melatonin in the human body.

It is therefore an object of the present invention to provide a device for automatic adjustment of the dose of melatonin and/or delivery of melatonin which is for most applications capable to respond to the changes, especially changes in biological functions over time.

This object is solved by a device according to claim 1 of the present invention. Accordingly, a device for automatic adjustment of the dose of melatonin and/or delivery of melatonin is provided, comprising
a) a measuring means, which measures at least one body parameter of a patient for at least >1 measuring cycle;
b) an adapting means, which adapts and/or changes the dose of melatonin according to the measuring data of the measuring means;
c) a delivery device for delivering melatonin to a person to be treated and/or a user of the device.

The term “adapting and/or changing” especially means and/or includes that the dose of melatonin is adapted and/or changed in order to reach a desired sleep pattern and/or interval.

By doing so, at least one of the following advantages is achieved for most of the applications within the present invention:
For a wide range of applications within the present invention, no initial longer term measurements are needed that have to be analysed before the therapy can start. This allows for a wide range of applications within the present invention to speed-up the therapy process providing an earlier start;
For a wide range of applications within the present invention, less side effects can be noticed;
For a wide range of applications within the present invention the therapeutic dose may be increased to increase treatment efficacy;
For a wide range of applications within the present invention, the drug amount is automatically adjusted upon the patient's endogenous hormone production;
For a wide range of applications within the present invention, the drug amount gradually converges to the desired outcome (i.e. sleep interval).

According to a preferred embodiment of the present invention, the at least one body parameter includes body temperature, core body temperature, skin surface temperature, activity, melatonin level, cortisol level, heart rate, breathing frequency.

According to a preferred embodiment of the present invention, the adapting means adapts and/or changes the dose of melatonin in order to reach a selected day/night activity ratio. In most cases this ratio has shown to be approximately 100:5 to 100:20 for a wide range of applications within the present invention.

According to a preferred embodiment of the present invention, the device comprises further a normalizing means to generate at least one normalized curve out of the data measured by the measuring means.

The term “normalized curve” means and/or includes especially that from the data derived from the measuring cycles, the normalizing curve is calculated by the equation:

Z=(X−mean(X))/standard deviation*100%
with X (also written as Xₜ) being the body parameter and mean(X) being the mathematical mean of Xₜ over a defined period. It should be noticed that usually X may have both positive and negative values.

However, in case the first body parameter includes activity, for a wide range of applications within the present invention the normalizing curve is calculated as follows:

Taking the average diurnal activity, taking the actual average (e.g. measured in an interval of 10 to 30 minutes) activity and dividing the actual average activity by the average diurnal activity.

According to a preferred embodiment of the present invention, the daily dose and/or dose for a certain time period of melatonin is set as to be

previous dose+Δmelatonin [Δm],
with the previous dose being the daily dose of the previous day or time period, whereby Δm is changed and/or set each time period, at least every 14 days, preferably at least every week, but most preferably daily (or for the next time period) according to the data of the measuring means.

It should be noted that the term “changed and/or set according to” does not mean that Δm is changed on the basis of the measuring means alone; however, according to the preferred embodiment, the data of the measuring means are taken into account when changing and/or setting Δm.

According to a preferred embodiment of the present invention, Δm is ≦50 μg/day, preferably ≦40 μg/day. This has been shown to be best suitable for a wide range of applications within the invention.

According to a preferred embodiment of the present invention, |Δm| is ≧0% and ≦80% of the maximum dose change Δmₘₐₓ. By doing so, it has been shown for a wide range of applications within the present invention that “overdosing” due a too high Δm can be avoided.

According to a preferred embodiment of the present invention, Δm is set as

Δm≧[Δmₘₐₓ*(ratio−a)/(1−a)]*0.8 and ≦[Δmaxₘₑₗₐₜₒₙᵢₙ*(ratio−a)/(1−a)]*1.2
with Δmₘₐₓ being the maximum dosage change of melatonin (preferably ≦50 μg/day more preferred ≦40 μg/day),
ratio being the actual ratio between nocturnal and diurnal activity and
a being the desired ratio between nocturnal and diurnal activity (≦0.25, corresponding with a day/night activity of 100:25, but preferably ≦0.10).

According to another embodiment of the present invention, the amount of melatonin delivered is decreased when the ratio and a have differed ≦0.1 (i.e. that |ratio−a| is ≦0.1), preferably ≦0.05 for at least one measurement cycle, preferably for at least 5 measurement cycles. By doing so, it is possible for a wide range of applications within the present invention to smoothen the adjustment to the preselected ratio and limit melatonin delivery when the endogenous production takes over such as in the treatment of the delayed sleep phase syndrome.

According to a preferred embodiment of the present invention, the decrease in daily melatonin is ≦20% per day and ≧5% per day of the dosage of the previous day until m (melatonin dosage) is ≦2.5 μg/day, preferably ≦1.0 μg/day. This range has been shown within a wide range of applications of the present invention to be most effective while lessening the probability of relapses.

According to a further embodiment of the present invention, while gradually decreasing the daily melatonin dosage, the measuring means is still used and the adapting means becomes active again once the |ratio−a| is no longer ≦0.1.

According to a preferred embodiment of the present invention, when the melatonin dosage drops below 2.5 μg/day, preferably below 1.0 μg/day, the dosage is set to zero.

According to an embodiment of the present invention, the device furthermore comprises a storing means for storing data on the daily melatonin dose, profile and/or activity. This has shown to be advantageous for a wide range of applications within the present invention. According to an embodiment of the present invention, the data stored can be read by the user only, according to a further embodiment of the present invention, the data stored can be read by the user and/or other persons e.g., a physician.

According to an embodiment of the present invention, the stored data can be used to stop the delivery of melatonin when the dosing amount has exceeded a certain preset threshold and/or the effectiveness of the melatonin treatment is below a certain other threshold.

According to an embodiment of the present invention, the stored data can be used to provide information that the delivery of melatonin has stopped after the device slowly decreased the daily amount of melatonin delivery.

According to a preferred embodiment of the present invention, the delivery of melatonin occurs along a drug delivery profile. By doing so it has been shown for a wide range of applications within the present invention that problems and/or inefficacies due to catabolic mechanisms of melatonin in the human body can be avoided or at least be reduced.

According to a preferred embodiment of the present invention, the drug delivery profile includes an increasing delivery phase whereby the amount of delivered melatonin increases with ≧1% and ≦12% per hr, preferably 5% and ≦10% per hr of the total daily melatonin dosage.

According to a preferred embodiment of the present invention, the drug delivery profile includes that the maximum delivery of melatonin in 1 hr is ≧10% and ≦30% of the total daily melatonin dosage. According to a preferred embodiment of the present invention, the drug delivery profile includes a decreasing delivery phase whereby the amount of delivered melatonin decreases with ≧1% and ≦12% per hr, preferably 5% and ≦10% per hr of the total daily melatonin dosage.

According to a preferred embodiment of the present invention, the adapting means is able to take into account information supplied by the person to be treated and/or a user of the device. Such information may include—but not be limited to—the judgement of the user of its own sleep pattern, a desired sleep interval and/or information of a physician concerning the status of the person to be treated.

According to a preferred embodiment of the present invention, the device comprises an input means for inputting the data from the person to be treated and/or a user of the device. These input means may include—but not be limited to—buttons or keys, interfaces for communication with e.g. computers in hospitals or doctor's surgery and/or means for wireless communication.

According to a preferred embodiment of the present invention, the delivery device comprises a patch or has a patch-like build-up. This has proved to be beneficial for a wide range of applications especially for users and/or patients who suffer from DSPS or other syndromes, which are more of a temporary nature.

According to a different preferred embodiment of the present invention, the delivery device comprises an implant or has an implant-like build-up. This has proved to be beneficial for a wide range of applications especially for users and/or patients who suffer from chronic sleep disturbances, e.g. in connection with Alzheimer.

The present invention furthermore relates to a method for the controlled release of drugs, comprising the steps of
a) measuring at least one body parameter of the patient for at least ≧1 measuring cycle;
b) adapting and/or changing the dose of melatonin on account of the measured data of the measuring means;
c) delivering melatonin to a person to be treated and/or a user of the device.

A device and/or a method according to the present invention may be of use in a wide variety of systems and/or applications, amongst them one or more of the following:
medical devices for the automatic adjustment of and/or delivery of melatonin;
Jet-lag treatment devices.

The aforementioned components, as well as the claimed components and the components to be used in accordance with the invention in the described embodiments, are not subject to any special exceptions with respect to their size, shape, material selection and technical concept such that the selection criteria known in the pertinent field can be applied without limitations.

### BRIEF DESCRIPTION OF THE DRAWINGS

Additional details, features, characteristics and advantages of the object of the invention are disclosed in the dependent claims, the figures and the following description of the respective figures, tables and examples, which—in an exemplary fashion—show one example of a melatonin treatment (i.e. dosage change) according to the invention.

FIG. 1 shows four diagrams of activity against time of activity during the day prior to melatonin treatment, on day 1 and 2 of melatonin treatment and a desired idealized activity curve (“day x”);

FIG. 2 shows three diagrams of activity against time of activity during the day prior to melatonin treatment (same as in FIG. 3), on day 3 of melatonin treatment and a desired idealized activity curve (“day x”, same as in FIG. 1);

FIG. 3 shows three diagrams of delivery of melatonin against clock time showing the three drug delivery profiles of the melatonin treatment of day 1 to 3;

FIG. 4 shows a schematic partial cut-out side view of a delivery device according to a first embodiment of the present invention implemented in a watch;

FIG. 5 shows a top view of the device of FIG. 4 approximately along line II-II in FIG. 4;

FIG. 6 shows a flow chart of an implementation of a method according to a first embodiment of the present invention.

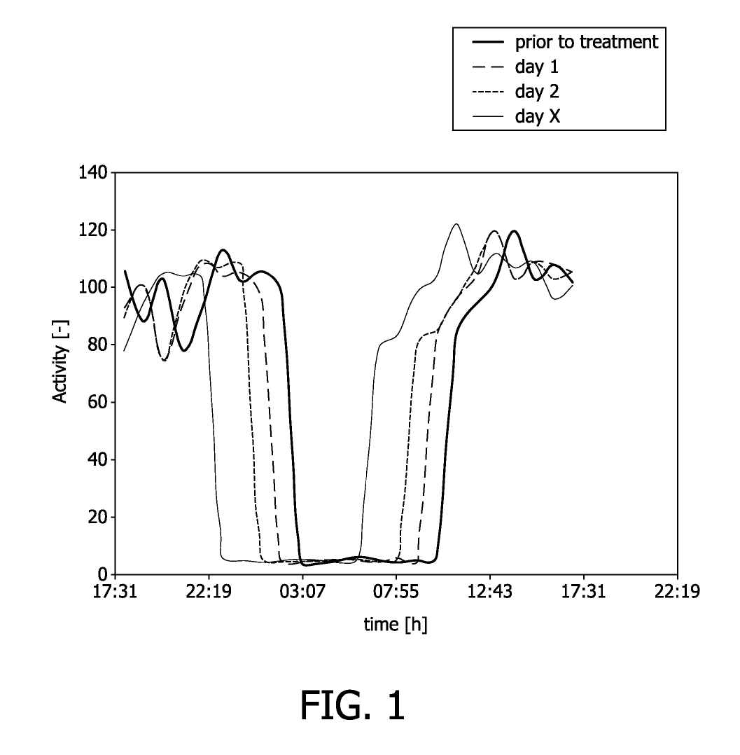

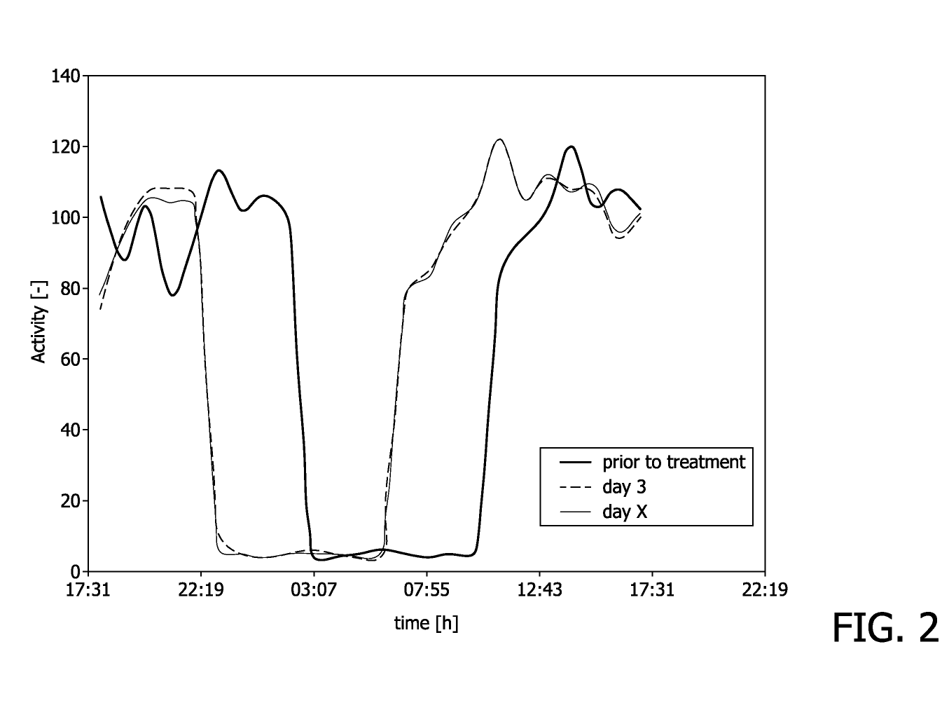

FIG. 1 shows four diagrams of activity against time of activity during the day prior to melatonin treatment, on day 1 and 2 of melatonin treatment and a desired idealized activity curve (“day x”), whereby the daily dose of melatonin was changed according to one embodiment of the present invention. It should be noted that the datapoints in FIGS. 1 and 2 have been averaged per hour (so that one datapoint is given for every hour).

The desired period of sleep was set from 23:00 to 7:00, however, prior to treatment the activity curve was clearly shifted with activity until about 3:00 and rising at about 11:00 (This data was used from a person suffering from delayed sleep phase syndrom (DSPS)).

FIG. 2 additionally shows the activity curve of day 3. It can clearly be seen that the desired idealized curve (“day x”) is approximated very well already on the third day of treatment.

The data of FIG. 1 and 2 is given in the table I hereafter

TABLE I
Normalized average hourly activity

| Clock | prior to | | | | |
|---|---|---|---|---|---|
| time | treatment | Day 1 | Day 2 | Day 3 | “Day X” |
| 18:00 | 106 | 90 | 93 | 74 | 78 |
| 19:00 | 88 | 101 | 100 | 96 | 95 |
| 20:00 | 103 | 75 | 74 | 107 | 105 |
| 21:00 | 78 | 97 | 95 | 108 | 104 |
| 22:00 | 93 | 110 | 108 | 106 | 103 |
| 23:00 | 113 | 104 | 107 | 12 | 7 |
| 00:00 | 102 | 105 | 108 | 5 | 5 |
| 01:00 | 106 | 96 | 6 | 4 | 4 |
| 02:00 | 99 | 5 | 5 | 5 | 5 |
| 03:00 | 5 | 5 | 4 | 6 | 5 |
| 04:00 | 4 | 4 | 5 | 5 | 5 |
| 05:00 | 5 | 6 | 6 | 4 | 4 |
| 06:00 | 6 | 5 | 5 | 5 | 6 |
| 07:00 | 5 | 4 | 4 | 76 | 78 |
| 08:00 | 4 | 6 | 5 | 85 | 83 |
| 09:00 | 5 | 5 | 80 | 96 | 98 |
| 10:00 | 6 | 81 | 85 | 105 | 104 |
| 11:00 | 83 | 96 | 96 | 122 | 122 |
| 12:00 | 94 | 104 | 108 | 105 | 105 |
| 13:00 | 102 | 120 | 120 | 111 | 112 |
| 14:00 | 120 | 103 | 103 | 108 | 107 |
| 15:00 | 103 | 109 | 109 | 107 | 109 |
| 16:00 | 108 | 103 | 108 | 94 | 96 |
| 17:00 | 102 | 106 | 106 | 100 | 101 |

The initial melatonin dosage (i.e. the dosage of day 1) was 20 μg. Using the formula:

Δm≧[Δmₘₐₓ*(ratio−a)/(1−a)]

Δmₘₐₓ is set at 40 μg/day

Δm for day 2 was calculated to be 19 μg, Δm for day 3 was calculated to be 13 μg. The overall daily dosage for day 2 was therefore 39 μg, for day 3 it was 52 μg.

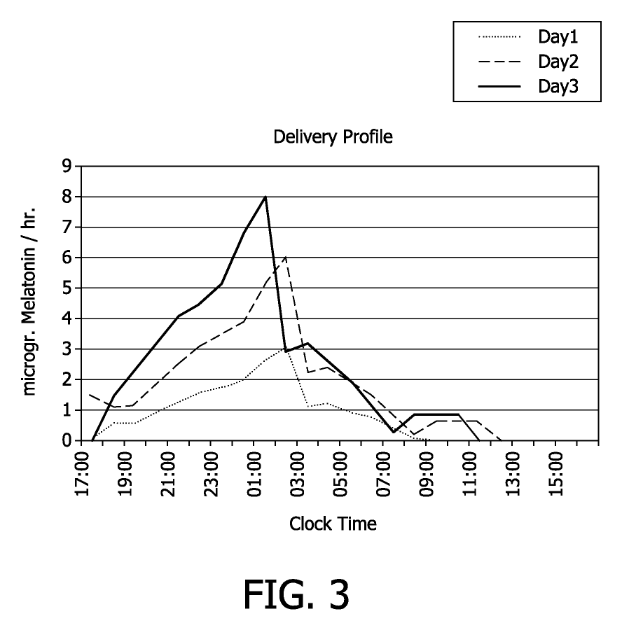

FIG. 3 shows the drug delivery profiles of the melatonin dosage of days 1 to 3. As described above, this delivery profile includes an increase of 1-12% (of the total daily melatonin dosage), until about 15% of the daily dose is given in one hour; after that the hourly dose is lowered by 1-12% per hour.

The data of FIG. 3 are given in Table II hereafter.

TABLE II
Melatonin drug delivery profile (μg/h)

| Clock | | | |
|---|---|---|---|
| time | Day 1 | Day 2 | Day 3 |
| 17:00 | 0 | 0 | 1.49 |
| 18:00 | 0.58 | 1.12 | 1.5 |
| 19:00 | 0.58 | 1.13 | 2.41 |
| 20:00 | 0.93 | 1.81 | 3.3 |
| 21:00 | 1.27 | 2.49 | 4.1 |
| 22:00 | 1.58 | 3.09 | 4.48 |
| 23:00 | 1.73 | 3.38 | 5.14 |
| 00:00 | 1.98 | 3.88 | 6.82 |
| 01:00 | 2.63 | 5.14 | 7.95 |
| 02:00 | 3.07 | 6 | 2.94 |
| 03:00 | 1.14 | 2.22 | 3.2 |
| 04:00 | 1.23 | 2.41 | 2.55 |
| 05:00 | 0.98 | 1.92 | 2.02 |
| 06:00 | 0.78 | 1.52 | 1.08 |
| 07:00 | 0.42 | 0.82 | 0.28 |
| 08:00 | 0.11 | 0.21 | 0.86 |
| 09:00 | 0 | 0.65 | 0.86 |
| 10:00 | 0 | 0.65 | 0.86 |
| 11:00 | 0 | 0.65 | 0 |
| 12:00 | 0 | 0 | 0 |
| 13:00 | 0 | 0 | 0 |
| 14:00 | 0 | 0 | 0 |
| 15:00 | 0 | 0 | 0 |
| 16:00 | 0 | 0 | 0 |

Please note that the delivery started on Day 3 an hour earlier than in Day 1 and Day 2

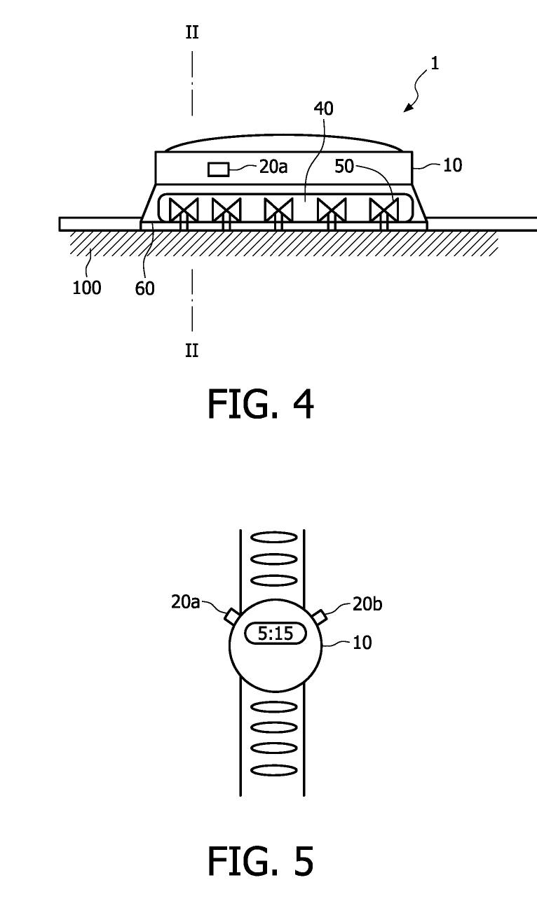

FIG. 4 shows a schematic partial cut-out side view of a delivery device 1 according to a first embodiment of the present invention implemented in a watch 10. The upper part of the watch case 10 is shown in front view, whereas the lower part (i.e. those parts referring to the numerals 40-60) is shown in cut-out view.

The device (beside the watch case 10, which also comprises the sensors and computing units for the device) 1 comprises a drug reservoir 40, from which the melatonin may be delivered to the skin 100 of the user and/or the person to be treated by several controllable valves 50. The device 1 is furthermore equipped with an antigliding material 60 located in proximity to the drug reservoir 40 in order to ensure the contact between the drug reservoir 40 and the skin 100.

It should be noted that instead of the valves 50, also further means might be possible to deliver the melatonin. According to one embodiment of the present invention (not shown in the Figs), the device uses electrophoresis to provide active delivery of melatonin.

FIG. 5 shows a top view of the device of FIG. 4 approximately along lineII-II in FIG. 4. As can be seen in FIG. 5, the watch case 10 is equipped with several input means 20a, 20b (here in the form of buttons) to set e.g. the desired sleep interval.

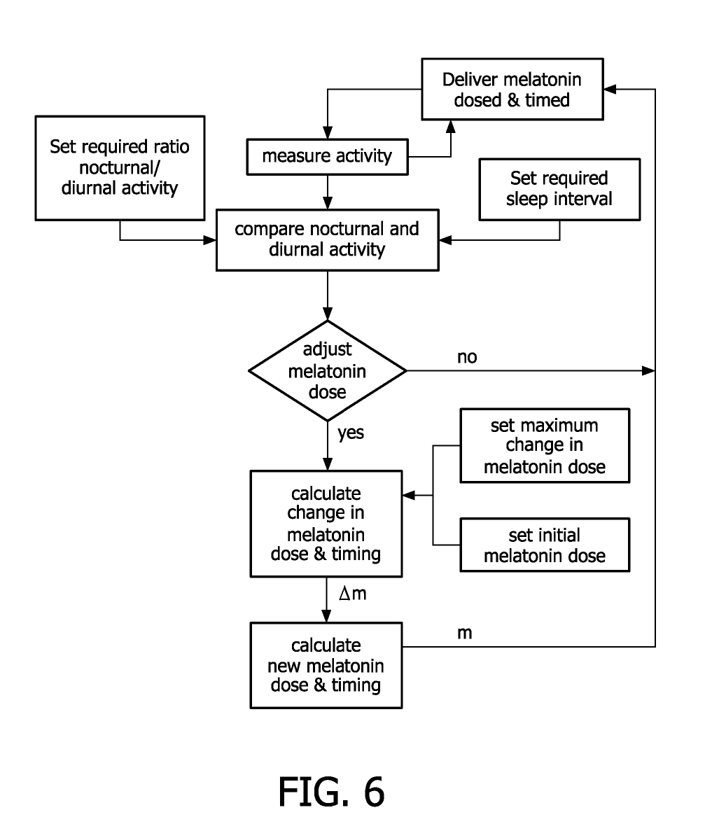

FIG. 6 shows a flow chart of an implementation of a method according to a first embodiment of the present invention.

As can be seen from FIG. 6, at first the melatonin is delivered (as expressed by the box “Deliver Melatonin dosed & timed”) and the activity is measured (“measure activity”). The measurement of the activity may occur while the melatonin is being delivered as well as after the delivery (as indicated by the “reverse arrow”).

Subsequently, the nocturnal and diurnal activity is compared by a comparing means. This comparing means also takes into account possible settings of the desired ratio between the nocturnal and diurnal activity and/or the required sleep interval.

In case the melatonin dose does not need to be adjusted (path “no” in the flow chart), the melatonin dosage and timing is continued without a change.

In case the melatonin dose needs to be adjusted, the change in melatonin dose and/or timing is calculated, taking into account possible settings in the maximum dosage of the melatonin dose as well as the initial melatonin dose.

From this change (Δm), the new melatonin dose & timing is calculated.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A device for automatic adjustment of the dose of melatonin and/or delivery of melatonin, comprising
a) a measuring means, which measures at least one body parameter of a patient for at least ≧1 measuring cycle;
b) an adapting means, which adapts and/or changes the dose of melatonin according to the measuring data of the measuring means;
c) a delivery device for delivering melatonin to a person to be treated and/or a user of the device.

2. The device according to claim 1, wherein the at least one body parameter includes body temperature, core body temperature, skin surface temperature, activity, melatonin level, cortisol level, heart rate, breathing frequency.

3. The device according to claim 1, wherein the daily dose of melatonin is set to be
previous dose+Δmelatonin [Δm],
with previous dose being the daily dose of the previous day, whereby Δm is changed and/or set each time period, at least every 14 days, preferably at least every week, but most preferably daily (or for the next time period) in accordance with the data of the measuring means.

4. The device according to claim 1, wherein Δm is ≦50 μg/day, preferably ≦40 μg/day

5. The device according to claim 1 wherein Δm is set as
Δm≧[Δmmax*(ratio−a)/(1−a)]*0.8 and ≦[Δmaxmelatonin*(ratio−a)/(1−a)]*1.2
with Δmmax being the maximum dosage change of melatonin,
ratio being the actual ratio between nocturnal and diurnal activity, and
a being the desired ratio between nocturnal and diurnal activity.

6. The device according to claim 1, whereby the amount of melatonin delivered is decreased when the ratio and a have differed ≦0.1 (i.e. that |ratio−a| is ≦0.1), preferably ≦0.05 for at least one measurement cycle, preferably for at least 5 measurement cycles, the amount of decrease being preferably ≦20% per day and ≧5% per day of the dosage of the previous day until m (melatonin dosage) is ≦2.5 μg/day, preferably ≦1.0 μg/day.

7. The device according to claim 1 wherein the delivery of melatonin occurs along a drug delivery profile.

8. The device according to claim 1 wherein the adapting means is able to take into account information supplied by the person to be treated and/or a user of the device.

9. A method for the controlled release of drugs, comprising the steps of:
a) measuring at least one body parameter of the patient for at least ≧1 measuring cycle;
b) adapting and/or changing the dose of melatonin according to the measuring data of the measuring means;
c) delivering melatonin to a person to be treated and/or a user of the device.

10. A system incorporating a device according to claim 1 and being used in one or more of the following applications:
Medical devices for the automatic adjustment of and/or delivery of melatonin;
Jet-lag treatment devices.
